(19) United States
(12) Patent Application Publication
(10) Pub. No.: US 2022/0107307 A1
(43) Pub. Date: Apr. 7, 2022

(54) UTILIZATION OF BLOOD COMPONENT-CONTAINING SOLUTIONS FOR BIOPROSTHETIC VALVE MANUFACTURE AND TESTING

(71) Applicant: The Trustees of Columbia University in the City of New York, New York, NY (US)

(72) Inventors: Giovanni Ferrari, Hastings on Hudson, NY (US); Antonio Frasca, Bronx, NY (US)

(21) Appl. No.: 17/499,511

(22) Filed: Oct. 12, 2021

Related U.S. Application Data
(63) Continuation of application No. PCT/US20/27619, filed on Apr. 10, 2020.
(60) Provisional application No. 62/972,754, filed on Feb. 11, 2020.
(60) Provisional application No. 62/860,670, filed on Jun. 12, 2019.
(60) Provisional application No. 62/833,450, filed on Apr. 12, 2019.

(51) Int. Cl. G01N 33/53; A61F 2/24; G01N 33/68; G01N 33/66

(52) U.S. Cl. CPC G01N 33/5306; G01N 33/66; G01N 33/68; A61F 2/2472

(57) ABSTRACT

Compositions and methods for testing BHV for performance or longevity are provided.

### CROSS-REFERENCE TO RELATED APPLICATIONS

The present application is a Continuation of International Application No. PCT/US20/27619 filed 10 Apr. 2020; which claims the benefit of U.S. Provisional Application Ser. Nos. 62/972,754 filed 11 Feb. 2020, 62/860,670 filed 12 Jun. 2019, and 62/833,450 filed 12 Apr. 2019; each of which are incorporated herein by reference in its entirety.

### STATEMENT REGARDING FEDERALLY SPONSORED RESEARCH OR DEVELOPMENT

This invention was made with government support under HL122805, HL143008 and HL007343 awarded by the National Institutes of Health. The government has certain rights in the invention.

### MATERIAL INCORPORATED-BY-REFERENCE

Not Applicable.

### BACKGROUND OF THE INVENTION

Heart valve disease is a common disorder, affecting millions worldwide, and at present cannot be treated medically. Surgery is the only option for severe heart valve disease, and can involve either repair or more commonly the use of a prosthetic heart valve. Bioprosthetic heart valves (BHV) fabricated from glutaraldehyde crosslinked heterografts, such as bovine pericardium (BP) or porcine aortic valves (PAV), are widely used to treat severe heart valve disease. BHV are preferred to mechanical prostheses because BHV in general have a lower risk of thrombo-embolic events, and BHV patients typically do not require anticoagulants. BHV at this time are either surgically implanted or deployed with transcatheter techniques. Surgical BHV are known to deteriorate after 10-15 years post-operation, most often due to structural valvular degeneration (SVD), frequently involving calcification. SVD occurs regardless of the heterograft material, requiring prosthesis replacement.

Calcification is by far the best established mechanism associated with SVD, wherein it is considered to contribute to tissue stiffening and functional stenosis as well as regurgitation due to gross leaflet deformation. Non-calcific SVD mechanisms, that account for an estimated 25% or more of BHV failures, have been investigated to a lesser extent. Bacterial endocarditis represents a relatively uncommon, but catastrophic cause of SVD, with high morbidity and mortality.

Compositions and methods for protecting BHV from SVD mechanisms, including non-calcific SVD mechanisms, would thus represent an important advancement in the field.

### SUMMARY OF THE INVENTION

The present disclosure provides a method of testing performance or durability of a bioprosthetic heart valve, the method comprising testing the performance or durability of the bioprosthetic heart valve in a pulse duplicator or cyclical fatigue tester in a solution containing serum albumin or a glycation precursor. In certain embodiments performance of the bioprosthetic heart valve is tested in a pulse duplicator, while in other embodiments durability of the bioprosthetic heart valve is tested in a cyclical fatigue tester. In certain embodiments, the testing is performed in both a pulse duplicator and in a cyclical fatigue tester. In certain embodiments the serum albumin or glycation precursor is present in the solution in a physiological concentration. In certain embodiments the glycation precursor is glucose, fructose, galactose, sucrose, maltose glyoxal, methylglyoxal, 3 deoxyglucoson, glycolaldehyde, ascorbic acid, or any other physiologically relevant precursor. In certain embodiments the solution comprises from greater than zero to about 25% serum albumin. In certain embodiments the serum albumin is human serum albumin. In certain embodiments greater than zero to about 30% of the serum albumin is glycated. In certain embodiments an alternative physiologically circulating protein is substituted for serum albumin at correspondingly relevant concentrations.

In certain embodiments of the disclosed methods, body fluid components associated with alternative biochemical pathways and disease processes are substituted for glycation precursors or added with glycation precursors for the modeling of the effects of said alternative pathways and processes on valve and bioprosthetic tissue function and durability. In particular embodiments the alternative disease process is calcification. In some embodiments the body fluid components associated with alternative biochemical pathways and disease processes include, but are not limited to, metal ions, cholesterol and lipid nanoparticles, or enzyme cofactors. In additional embodiments the solution containing body fluid component molecules is applied as a pre-treatment of the bioprosthetic heart valve or bioprosthetic tissue before testing.

The present disclosure also provides a kit for testing performance or durability of a bioprosthetic heart valve, comprising a buffered or non-buffered saline solution and serum albumin. In certain embodiments the kit further comprises a glycation precursor. In certain embodiments the serum albumin is human serum albumin. In certain embodiments greater than zero to about 30% of the serum albumin is glycated. In some embodiments the kit further comprises a body fluid component associated with an alternative biochemical pathway or disease process. In various embodiments the body fluid component associated with an alternative biochemical pathway or disease process includes, but are not limited to, a metal ion, a cholesterol and lipid nanoparticle, or an enzyme cofactor. In other embodiments the alternative disease process is calcification.

### DESCRIPTION OF THE DRAWINGS

Those of skill in the art will understand that the drawings, described below, are for illustrative purposes only. The drawings are not intended to limit the scope of the present teachings in any way.

FIG. 1 shows a simplified schematic of bioprosthetic tissue, exemplified by glutaraldehyde-fixed bovine pericardium. Glutaraldehyde crosslinks that impart tissue durability are assumed and not shown (though it is notable that these crosslinks occupy a significant proportion of tissue lysines). These tissue are most often ˜90% collagen, for which functionally-significant glycation susceptibility is well-characterized. Thus, only collagen is shown for simplicity. Collagen is arranged in partially-aligned stacks. The fibers dissipate force during tissue biomechanical activity by sliding past each other and by stacks compressing and relaxing in the Z dimension, indicated by arrows.

FIG. 2 shows a simplified schematic of tissue alteration during implantation. Glutaraldehyde-fixed bioprosthetic heart valve biomaterials exhibit essentially no resistance to infiltration of circulating serum albumin and glycation precursors in vitro. This is likely true for many, if not all, proteins, other macromolecules, and small molecules in the physiologic fluids to which they are exposed during clinical implantation. Thus, immediately upon implantation, serum albumin and glycation precursors—as well as associated and separate other molecules and the solvent itself, water—infiltrate into the tissue. This immediately alters the dry mass of the tissue, thus affecting tissue biomechanics.

Further, glycation can cause permanent incorporation of infiltrated proteins into the tissue via crosslinks between the infiltrated proteins and the protein molecules of the tissue matrix, thus permanently inculcating the properties of those proteins into the tissue. In the specifically prominent case of serum albumin, its high oncotic pressure capability can tightly bind water molecules, driving water into the tissue and mediating its thickening over time. Similarly, albumin is known to bind lipids, calcium, and various other molecules, whose infusion into valve tissue will be influenced and possibly exacerbated by the presence of albumin. Over time, glycation crosslinking and albumin infusion could drive the galloping accumulation of infiltrated blood components into the tissue, modifying tissue performance and ultimate durability.

FIG. 3 shows the results of assay of lyophilized dry mass increase in 2 cm-diameter discs of glutaraldehyde-fixed bovine pericardium due to exposure to and infiltration of bovine (BSA) or human serum albumin (HSA) in glutaraldehyde-fixed bovine pericardial tissue discs. Discs are rinsed in phosphate-buffered saline (PBS) overnight and desalted via repeated washes in distilled H₂O prior to weighing before and after incubation. Results show ˜4-5% increases in tissue dry mass due to 24-hour exposure at 37° C. to bovine or human serum albumin with or without 50 mM glycation precursor glyoxal.

FIG. 4 shows a time course of human serum albumin mass uptake according to the same protocol as in FIG. 3. Data show mass uptake after incubation at 37° C. in PBS or PBS supplemented with 5% (w/v) HSA alone or together with 50 mM glyoxal, a prominent physiologic precursor of glycation. Incubation times assayed are 24 hours, 7 days, and 28 days. Results indicate that incubation in albumin alone generates an apparent diffusive equilibrium that gives rise to a stable ˜4-5% tissue dry mass increase in glut-BP, which displays a slight but steady increase over time due to the condensation with tissue proteins of Amadori-modified albumin present in the human-isolated HSA stock, while co-incubation with glycation precursor drives a galloping accumulation that steadily increases tissue dry mass, leading to a ˜13% dry mass increase after 28 days of incubation.

FIG. 5 shows electron microscopy of glutaraldehyde-fixed bovine pericardium incubated at 37° C. in phosphate-buffered saline (PBS, left panels), PBS supplemented with 5% (w/v) HSA alone (center panels) or together with 50 mM glyoxal, a prominent physiologic precursor of glycation (right panels). Images reveal an abundance of inter-collagen particulates in albumin-exposed conditions and the emergence of non-collagen fiber-like aggregates in glyoxal-albumin co-incubated tissue.

FIG. 6 shows electron microscopy glutaraldehyde-fixed bovine pericardium incubated at 37° C. in phosphate-buffered saline (PBS), PBS supplemented with 5% (w/v) HSA alone (HSA), PBS together with 50 mM glyoxal alone (glyoxal) or PBS with 50 mM glyoxal and 5% (w/v) HSA (Glyoxal+HSA) after a 1 day incubation, 14 day incubation or 28 day incubation. Results reveal the growth and organization of disruptive fiber-like aggregates in albumin-containing conditions and the association of inter-collagen fiber particulates and aggregates with disrupted collagen fiber surfaces.

FIG. 7 shows surgical bioprosthetic heart valve performance parameters evaluated by pulse duplication testing. Equivalent clinical-grade commercial BHV were incubated at 37° C. in either PBS or PBS supplemented with 5% HSA and/or 50 mM glyoxal for 35 days. Valves were removed from incubations for pulse duplicator testing after 1, 3, 7, 14, 21, 28, and 35 days and replaced to incubation in fresh solutions until the 35-day time point. Effective orifice area at maximum valve opening (left panel), positive pressure gradients across the valve (center panel), and fluid ejection velocity (right panel) are reported for each time point.

### DETAILED DESCRIPTION OF THE INVENTION

The present disclosure is based, at least in part, on the discovery that the accumulation of glycation products, including advanced glycation end products (AGEs) and concomitant incorporation of infiltrated blood proteins in BHV leaflets instigates tissue stiffening via crosslinking, tissue thickening, and host inflammatory responses via receptor signaling, and, furthermore, that these mechanisms contribute to BHV SVD.

Heart valve disease at this time can only be treated surgically, with either valve replacement or repair. Bioprosthetic heart valves (BHV), fabricated from glutaraldehyde fixed heterografts, such as bovine pericardium (BP) or porcine aortic valves (PAV), are widely used in both cardiac surgery and in transcatheter valve replacements. Despite outstanding short term outcomes, BHV dysfunction due to structural valve leaflet degeneration (SVD) develops over time, frequently necessitating device replacement. BHV are porous materials and lack any functional endothelium or surface barrier structure. As such, they are susceptible to insudation by blood biochemicals during clinical implantation. It has long been established that blood-derived small molecules, lipids, and macromolecules, such as the circulating calcium-binding proteins osteopontin and osteocalcin, infiltrate into BHV and affect processes related to valve functionality and performance. Recent studies showing that statin treatment reduces calcification, inflammatory cell invasion, and accumulation of inflammatory markers in BHV tissues further validate that the state of blood biochemistry influences and modifies BHV tissue.

Infiltration of blood components could be diffusive/transient or could lead to deposition into and permanent modification of BHV tissue. The above studies and others have shown that leaflet tissue is mineralized and oxidized by small molecules in the blood and that patient-derived macromolecules and even cells can infiltrate the tissue. Further, the infiltration/incorporation of blood components is known to inculcate the infiltrating molecules' properties to the BHV tissue, as in the case of the presence of calcium-binding proteins associating with BHV tissue mineralization and the presence of inflammatory stimulators associating with immune cell attack of the implant. Many of these processes even occur in the context of subcutaneous implantation model systems, wherein the BHV tissue is both immobile and only exposed to interstitial fluid rather than the full complexity of blood. Thus, BHV tissue and their properties are dynamically modified upon clinical implantation. Additionally, comorbidities that alter blood biochemistry, such as diabetes, are associated with more prevalent and earlier-onset valve degeneration and related complications.

BHV functionality consists of leaflet opening and closing mechanical activities that maintain pressure gradients, hemodynamics and flow rates, and cardiac waveforms at the relevant valve position. Functional performance is fundamentally susceptible to tissue degenerative processes during clinical implantation and exposure to physiologic conditions that contribute to structural valve degeneration (SVD), which culminates in functional failure and ultimately necessitates prosthetic valve replacement. SVD has historically limited BHV to an average lifespan between 10 and 15 years. Multiple mechanisms have been causatively implicated in BHV SVD. Most prominently among these are calcification, inflammatory response-induced tissue oxidation, fibrocalcific remodeling, and tissue thickening. Calcification is observed in the majority of SVD cases; however, 25% or more of SVD cases are not associated with calcification. These processes are biological in nature; thus, while they contribute to collagen network and extracellular matrix disruption in general, they differ from such issues of pure material fatigue occurring as a result of mechanical activity or material aging alone. Due to the nature of BHV biomaterials as non-living, metabolically inactive tissues, these processes must necessarily arise from patient-prosthesis interactions of biochemical, physiologic, and mechanical natures. As indicated above, infiltration by blood components and modification of BHV tissue are at least partially causative of many of these mechanisms. Particularly, leaflet thickening can only occur through the deposition and accumulation of blood molecules, given that no persisting metabolizing cells occupy the valve tissue. Therefore, processes that initiate upon clinical implantation via infiltration of blood components into BHV tissues contribute to ongoing valve performance and to ultimate valve durability.

Two particular blood components seem to play outsized roles in this regard. Serum albumin specifically and precursors of glycation in general exert key immediate and long-term synergistic effects on BHV tissue properties via infiltration. The inventors have observed in BHV explant samples the presence of advanced glycation end products (AGE), including carboxymethyl-lysine (CML) a ligand for the receptor for AGE (RAGE) and glucosepane, the most abundant AGE crosslink. The inventors have demonstrated that glycation is a key mechanism contributing to BHV degeneration.

The inventors have also determined that infiltration into the valve tissue and incorporation via glycation of blood proteins—most notably serum albumin—occurs in clinical valves, modifies tissue properties/valve performance, and contributes to valve degeneration. Albumin is by far the most abundant protein in the blood—accounting for over half of all blood proteins by mass; therefore, it is inherently uniquely important with regard to blood components in this respect. The inventors have demonstrated that the bioprosthetic tissues from which these valves are manufactured have no resistance to the infiltration of albumin from surrounding fluid; as a result, albumin begins to infiltrate into the tissue immediately upon exposure to a fluid containing it (i.e., blood, in the clinical context) and diffuses evenly throughout the depth of the tissue, both in vitro and in vivo. Albumin can be permanently incorporated into the valve tissue by glycation via physiologically-relevant precursors, such as sugars and sugar-derived dialdehydes. In this context, albumin incorporation has both glycation-related and glycation-independent effects on valve tissue and performance.

Current testing standards, as described by ISO 5840, only refer to the mimicry of blood conditions insofar as the modeling of blood viscosity; they do not consider any effects of blood biochemistry on the valve tissue or valve performance in general and are not aware of the significance to the alteration of valve tissue and functional performance or durability of glycation or serum albumin/protein infiltration.

Based on these findings, the present disclosure provides compositions and methods for improved performance and durability testing, as well as manufacturing, of BHV.

Bioprosthetic Heart Valves (BHV), Blood Component Infiltration and Structural Valve Degeneration (SVD)

Heart valve disease is a major clinical problem in both scope and severity. More than 500,000 people in the United States alone suffer from severe aortic stenosis, which, if left untreated, offers lower two-year survival rates than many metastatic cancers. The remaining 3 heart valves—particularly the mitral valve—are also subject to heart valve disease. In total, it is estimated that roughly 100,000,000 people worldwide suffer from the various heart valve diseases. Valve disease is most often treated by replacement with a prosthetic valve, but in some cases may be treated by native valve repair. In cases of valve repair, patches made of glutaraldehyde-treated bovine pericardium are often used to replace resected valve tissue. In cases of valve replacement, bioprosthetic heart valves (BHV) or mechanical valves may be employed. Nearly 300,000 aortic valve replacements occur worldwide each year, with 80,000 to 100,000 occurring in the United States.

Bioprosthetic heart valves are manufactured from glutaraldehyde-treated animal tissues dominated by bovine pericardium and porcine aortic valve tissues. BHV currently hold roughly 80% market share for valve replacements, and this proportion is increasing due to the greater versatility of these valves over mechanical valves. In particular, the advent of surgery-sparing transcatheter valve replacement approaches is driving the increased utilization of BHV. Notably, while mechanical valves are manufactured from non-porous materials such as pyrolytic carbon, BHV are porous materials and lack any functional endothelium or surface barrier structure. As such, they are susceptible to insudation by blood biochemicals during clinical implantation.

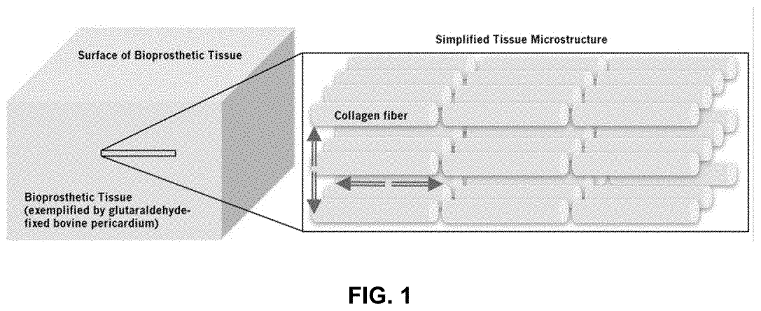

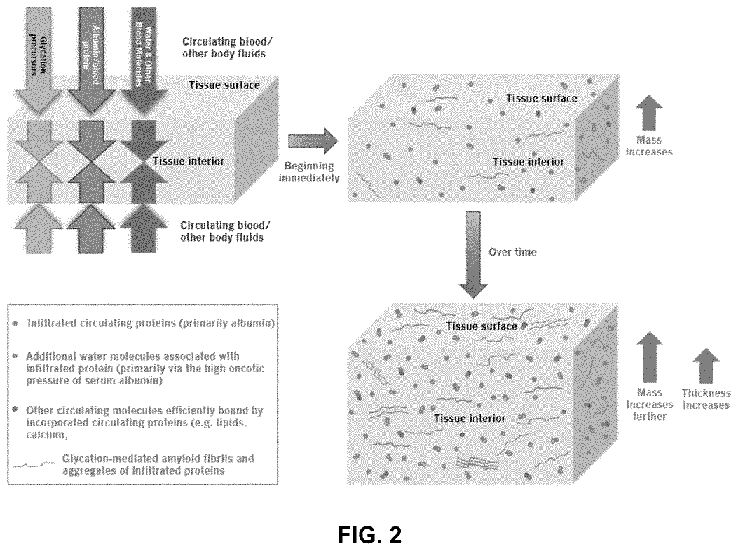

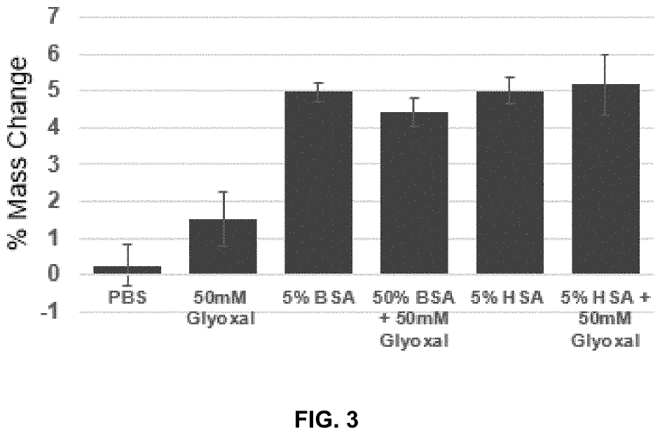

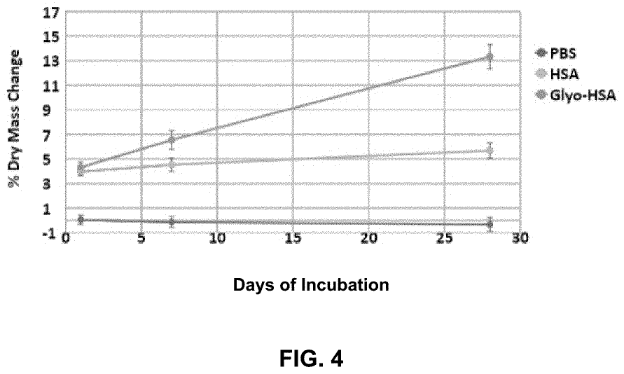

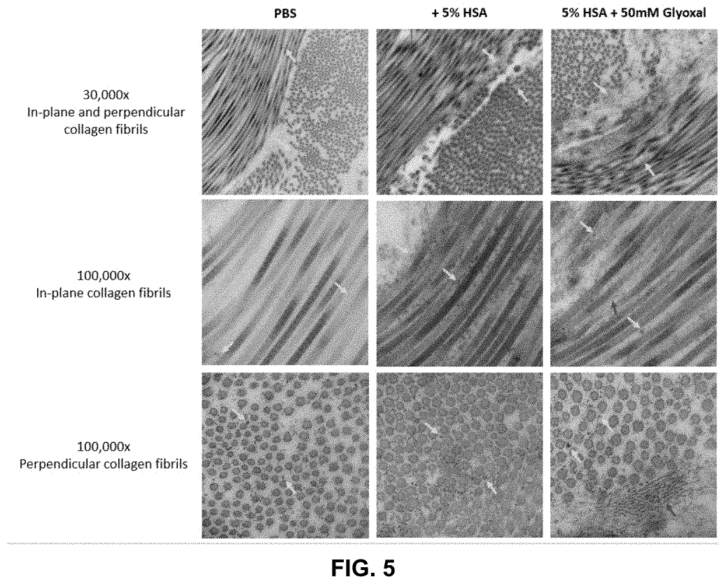

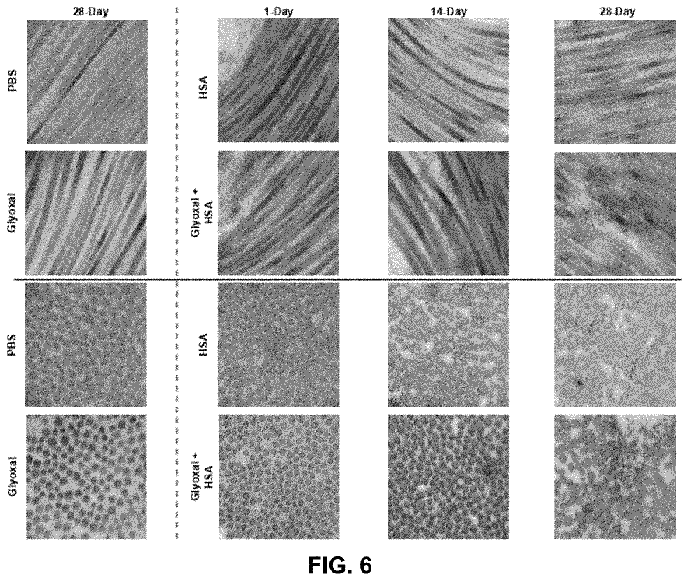

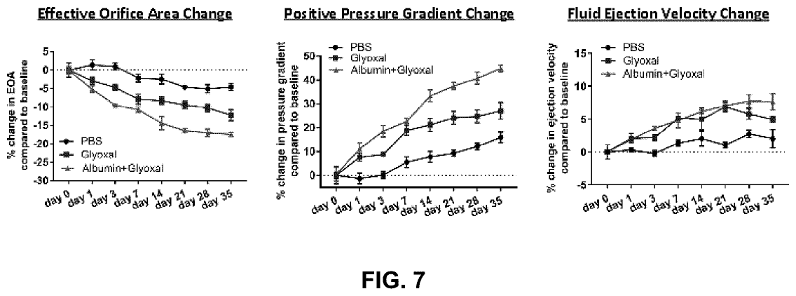

Glycation has not previously been significantly considered in SVD or the degeneration of bioprosthetic biomaterials in general. However, it is firmly established as functionally degenerating collagen in cardiovascular tissues. BHV biomaterials are composed predominantly of collagen type 1, with bovine pericardium being approximate 90% collagen. A simplified schematic of the base tissue structure is shown in FIG. 1. Additionally, it is established that glycation in collagenous vessel walls and solid tissues is reduced upon insulin treatment, which effectively lowers the concentration of circulating glycation precursors, suggesting that infiltration of circulating glycation precursors is causative of collagenous solid tissue glycation. Supporting the relevance of this mechanism to BHV, recent data have shown that BHV suffer higher incidence and earlier onset of SVD in diabetic patients. Correspondingly, infiltration of solid tissues is a fundamental feature of albumin function, and albumin has been shown studding collagen fibers in human and animal aortae by visualization techniques. FIG. 2 shows a simplified schematic of tissue alteration during implantation, and FIG. 3 shows immunohistochemistry of explanted failed valves and in vitro exposure to serum albumin with and without glycation precursors, which establishes the clinical relevance of albumin infiltration and glycation in BHV. The overlap of staining patterns suggests synergy between these mechanisms in two ways: 1. the exacerbation of long-term glycation by albumin deposition; 2: the permanent incorporation of infiltrated albumin (and, by extension, body fluid proteins in general) by glycation crosslinking. It has been proposed in the literature that infiltration of Amadori-albumin and subsequent resolution of Amadori products to crosslinking AGEs permanently fuse previously-circulating albumin to collagen fibers in myocardium and instigate chronic edema and fibrosis due to the high oncotic pressure of albumin. Compounding the ability of glycation precursors and glycated proteins to infiltrate collagenous tissues, BHV do not have biologically function endothelia or barrier layers, and efforts to produce such have repeatedly proved ineffectual. The presence of circulating calcium-binding proteins has previously been shown in BHV, and tissue thickening regularly observed in explanted BHV must require incorporation of blood components, given the lack of any living cells resident in BHV. In particular, incorporation of serum albumin by glycation crosslinking would be expected to be particularly instigative of tissue thickening due to the oncotic pressure effect and the providing to the tissue of additional residues capable of further glycation. Crosslinking of BHV tissue extracellular matrix structural fibers to each other and to infiltrated blood components could therefore provide core mechanisms of tissue thickening, collagen network disorganization and tissue stiffening—all hallmarks of SVD. Moreover, signaling AGEs formed on BHV tissues could instigate inflammatory attack and concomitant oxidation of the leaflet, thus providing a glycation-related mechanism giving rise to an additional hallmark of SVD.

Serum Albumin Infiltration and Glycation

Two particular blood components seem to play outsized roles in this regard. Serum albumin specifically and precursors of glycation in general exert key immediate and long-term synergistic effects on BHV tissue properties via infiltration. Serum albumin is significant among blood proteins with respect to infiltration into and modification of solid tissue properties due to both its abundance and its particular properties. Albumin accounts for more than half of all circulating protein by mass, exerts a uniquely high oncotic pressure, binds a variety of other circulating molecules including lipids and calcium, and is highly susceptible to glycation. Albumin readily infiltrates glutaraldehyde-fixed bovine pericardium (glut-BP), with tissue samples exposed to 5% human serum albumin (HSA) in phosphate-buffered saline for 24 hours exhibiting strong and uniform immunohistochemical staining for HSA (biorxiv.org/content/10.1101/2020.02.14.948075v1).

Correspondingly, infiltration of albumin induces a stable 4.5-5% increase in the dry mass of glut-BP throughout the depth of the tissue by 24 hours of exposure (FIG. 4). There is no difference in dry mass due to PBS alone compared to clear mass increases due to exposure to albumin. Comparison of results at different time points show infused albumin is retained through PBS and water washes and contributes to tissue mass, with increase evident within 1 hour of incubation and achieving an apparent saturation of 4.5-5% mass added by 24 hours. Co-incubation with glyoxal, a glycation precursor capable of generating protein-protein crosslinks, ultimately seems to drive albumin incorporation beyond the diffusive saturation point, exhibiting a mass uptake above 6% after 7 days of incubation and around 13% after 28 days of incubation. This progressive mass increase is likely driven by permanent incorporation of infiltrated albumin into the tissue by glycation crosslinks between the albumin and tissue structural proteins, effectively removing infiltrated HSA and its associated mass from the diffusive pool, driving additional diffusive infiltration and covalent incorporation.

These results suggest two things: first, that albumin infusion-induced mass increase is relevant to bioprosthetic valve performance immediately upon clinical implantation; and second, that circulating glycation precursors drive galloping accumulation of diffusively infiltrated albumin/protein over time, thus compounding any effects it has on valve durability. Thus, valve tests that takes these considerations into account will be much more representative of how the valves actually perform and endure under physiologic conditions.

FIG. 5 and FIG. 6 show electron microscopy of glutaraldehyde-fixed bovine pericardium incubated at 37° C. in phosphate-buffered saline (PBS) or PBS supplemented with 5% (w/v) HSA alone or together with 50 mM glyoxal, a prominent physiologic precursor of glycation. Collagen fibrils and particles in interstitial spaces are evident. 30,000× magnification images show regions in which collagen bundles containing fibrils oriented parallel to the image plane and fibrils oriented perpendicular to the image plane are adjacent to each other. Sets of 100,000× images highlight areas within collagen bundles with fibrils oriented either parallel or perpendicular to the image plane, respectively. Yellow (light) arrows indicate representative particulates. Some particulates are evident in untreated glut-BP, likely representing infiltrated circulating proteins deposited during the life of the donor bovine, during whose life the pericardium is constantly perfused with interstitial fluid. The abundance of these interfibrillar particulates is significantly increased upon in vitro exposure to albumin with or without glycation precursor. These results indicate that albumin (and likely circulating proteins in general) infiltrate the tissue not only on the more expectable level of the void spaces between collagen fiber bundles, but more pervasively into collagen fiber bundles themselves, interacting directly with collagen fibers. These infiltrated albumin particles may modify tissue sterics and stereolectronics, thus disrupting the dissipation of force during biomechanical activity by collagen fibril sliding and bending. Perhaps more important, though, are the effects of glycation precursor in infiltrated proteins. One day of co-incubation with glyoxal generates fibrous aggregates, as indicated by red (dark) arrows. These fibrous aggregates grow and interact with each other, generating their own fibrous network that seems to be interacting with collagen fibers within 28 days of incubation. Albumin is known to form fibrous amyloids upon glycation. Thus, the physiologically relevant simultaneous presence of infiltrating circulating albumin and glycation precursors can generate amyloid structures than can mediate longer-distance inter-collage crosslinks and concomitant tissue stiffening as well as more dramatically disrupt tissue matrix structure. Further, evident by 14 days of incubation and worsened by 28 days, infiltrated protein and glycated aggregates appear directly associated with and responsible for the disruption of collagen fiber surfaces, as indicated by their blurred/frayed appearance in in-focus images of fiber cross-sections (FIG. 6). These observations provide a mechanistic underpinning for functional disruption of tissue structure and biomechanics.

Albumin infiltration into solid tissues is associated with edema due to its high oncotic pressure; thus, albumin infusion is expected to increase functional wet tissue mass significantly beyond the 4-5% mass added by the albumin itself. Given that tissue mass directly affects biomechanical forces and stress, albumin infiltration will modify bioprosthetic valve leaflet motion and resultant performance parameters in the immediate term as well as influence valve durability in the long term. Additionally, because of its binding properties and central biological role as an arbiter of blood biochemistry, albumin infiltration is likely to influence accumulation of lipids, calcium, and other circulating proteins in clinically implanted BHV. Also, albumin is a large globular (approximately spherical) protein, compared to the thin and elongated fibrous nature of BHV tissue collagen, which is arranged in stacked layers of organized, parallel fibers; further, while collagen and glycosaminoglycans—two proteins accounting for nearly all of the BHV tissue material—are positively-charged, albumin is negatively-charged. This charge difference encourages close interaction of the proteins and modification of the stereoelectronic environment of the tissue proteins. These properties collectively can be expected to disrupt BHV tissue molecular organization by sterically, stereoelectronically, and osmotically disrupting collagen network alignment, normal interactions among tissue proteins, and resultant dissipation of forces during valve biomechanical activity. The inventors have demonstrated, using second-harmonic generation imaging, that collagen network alignment and fiber properties are disrupted via exposure to 5% albumin alone. Finally, albumin is highly susceptible to glycation.

Serum albumin is particularly susceptible to blood-based glycation due to its abundance and the number of glycation-susceptible residues each molecule contains. Canonical HSA harbors many sites for glycation: in vivo reports note glycation at 38 of its 59 lysine residues and 8 of its 24 arginine residues, and in vitro reports demonstrate glycation at all 59 lysines and 19 arginines as well as the N-terminus. Thus, 1-10% of albumin molecules in circulation at any given time are glycated in normoglycemic individuals. In diabetics, this proportion can reach above 30%. The typical lifespan of an individual albumin molecule in the body is roughly three weeks. For this reason, while glucose levels are used for short-term glycemic control monitoring in diabetics and glycated hemoglobin A1C—whose molecules have an average lifespan of 120 days—levels are used to indicate long-term control, glycated albumin levels are now commonly used to indicate mid-term glycemic control. The particular significance of the lifespan of albumin molecules in this regard lies in the timescale overlap with the stability of Amadori glycation intermediates. Because of the overlapping timescales, nearly all glycated albumin in circulation is in the Amadori form. Therefore, roughly 1-10% of all albumin molecules in normoglycemic individuals and up to 30% in diabetic individuals are Amadori-albumin. This is significant in the context of albumin infiltration into BHV due to the facilitation of glycation-mediated protein-protein crosslinking that permanently incorporates albumin into BHV tissues. As a further byproduct of this incorporation, albumin infuses the biomaterial with an abundance of glycation-susceptible residues. In particular, while many of the lysines in BHV biomaterial are blocked from glycation due to glutaraldehyde crosslinking, incorporated blood proteins supply unprotected lysines. This modifies the glycation landscape by enabling enhanced generation of particularly deleterious lysine-directed glycation products, such as the most important glycation crosslinker glucosepane and the most established inflammatory glycation product, N-carboxymethyl lysine. It also enables ongoing accumulation of blood proteins via crosslinking. The inventors' mass uptake data has shown that co-incubation of glutaraldehyde-fixed bovine pericardium in 5% albumin and 50 mM glyoxal for one week pushes the increase of tissue dry mass (and therefore albumin addition) beyond the apparent diffusive saturation point achieved by incubation in albumin alone. Thus, albumin infusion exacerbates glycation and glycation exacerbates albumin infusion, and this synergy between albumin infiltration and glycation can lead to progressive disruption of valve tissue, performance, and durability.

FIG. 7 shows the effects of albumin and glycation on valve performance in a pulse duplicator. Results show significant deterioration of effective orifice area, positive pressure gradients, and fluid ejection velocity in glyoxal-treated valve versus control, and these deteriorations are dramatically enhanced by co-incubation with albumin (only mildly enhanced in the case of ejection velocity).

Glycation Biochemistry and Biology

Glycation is a fundamental biochemical phenomenon defined by the non-enzymatic adduction of sugars and sugar-derived products to nitrogenous groups on proteins and amino acids. Glycation chemistry is both complex and convoluted. Glycation generally begins with nucleophilic attack by protein groups—most commonly nitrogens—on sugar carbonyl carbons, which include both aldehyde and ketone moieties. This generates key unstable imine intermediates including, most prominently, Schiff bases, which then undergo downstream resolution processes to stable products. In the case of glycation by intact sugars, this pathway of stable resolution of Schiff bases often involves Amadori rearrangement to ketoimines using sugar hydroxyl groups. Subsequently, a constellation of rearrangements, condensations, and further reactions generate myriad intermediates and biologically irreversible endpoint products, referred to as “advanced glycation end products” (AGEs).

Biologically relevant glycation precursors include, but are not limited to, glucose, fructose, sucrose, galactose, maltose, dextrose, glyoxal, methylglyoxal, 3-deoxyglucosone, glycolaldehyde, and ascorbic acid. More broadly, the considered precursor classes can be described as sugars (including hexoses and pentoses), sugar-derived reactive aldehydes and ketones, and small molecule metabolites and vitamins (such as ascorbic acid). The most common sites by far for physiologic glycation are lysine and arginine side chain nitrogens, with select other residues with potentially nucleophilic side chains contributing minorly to overall glycation. Glycation spontaneously occurs in normal physiology and is exacerbated in diabetes and metabolic diseases, due to the overabundance of sugar and highly-reactive sugar byproducts. The dozens of identified protein-bound AGEs include cell signaling adducts, such as N-carboxymethyl lysine (CML) and crosslinkers, such as the lysine-arginine crosslink glucosepane. Among glycation intermediates, so-called “Amadori” products are physiologically significant. These products represent the adduction of glycation precursors to proteins in a state capable of further condensation and are stable on the order of 1-2 months. Thus, glycation-mediated crosslinking between proteins, which would otherwise require a tri-molecular interaction, requires only two molecules and a more direct reaction pathway in the context of Amadori-proteins. Glycation spontaneously occurs in normal physiology and is exacerbated in diabetes and metabolic diseases, due to the overabundance of sugar and highly-reactive sugar byproducts. Circulating levels of several glycation precursors have been measured in human patients and are observed to be elevated in diabetics.

Glycation biochemistry is established in functional mechanistic detail as causative of tissue degeneration in a large variety of tissue types and diseases, including cardiovascular diseases, complications of diabetes, Alzheimer's disease, ocular diseases, kidney disease, metabolic syndrome, bone diseases, tooth decay, and aging. Prevailing themes in the functional causation of disease by glycation are the instigation of inflammatory processes by cell-signaling AGEs as well as the functionally-deleterious stiffening of structural and vascular tissues by crosslinking, which, in turn, instigates organ dysfunction. Glycation crosslinking and related functional tissue disruption generally impinge on extracellular matrix structural proteins and are particularly established with respect to collagen and collagenous tissues. For example, glycation crosslinking is causatively associated with various diabetic complications—including retinopathy and nephropathy—and arterial stiffening due to the crosslinking of vessel wall matrix proteins including collagen and elastin. Further, the disruption of the biomechanical properties and performance of collagen molecules and collagenous tissues by glycation is well established both in vitro and in vivo.

Protein glycation occurs in any situation in which proteins are exposed to glycation precursors—both within cells and in body fluids. Thus, soluble proteins in blood, lymph, and interstitial fluids are subject to glycation. In fact, levels of the glycated form of hemoglobin A1C are the accepted standard for monitoring longer-term glycemic levels in diabetics. It is also known that 1-10% of serum albumin—which accounts for more than half of all blood protein by mass—circulating at any given time is glycated in normoglycemic, healthy individuals; this proportion can reach 30% in diabetics. Glycated albumin is emerging as an improved marker over glycated HbA1C for many diabetic complications and is considered the standard marker for mid-term glycemic control in diabetics. Much of the effect of glycation on solid tissues is likely due to infiltration of glycation precursors and glycated proteins from body fluids rather than in situ-cell-based glycation, as evidenced by the reduction of glycation-induced diabetic complications by insulin treatment, which, in effect, lowers circulating glycation precursor levels by triggering their uptake by cells.

Proteins and lipids in body fluids have been shown to infiltrate into bioprosthetic tissues. These tissues are also known to be degraded by processes resultant from the infiltration of small molecules from body fluids. Amadori products are specifically important glycation intermediates because their stability timescale overlaps with that of many circulating proteins. In the context of blood protein infiltration into solid tissues, Amadori products enable direct crosslinking between infiltrating protein species and solid tissue proteins without further need for a third molecule, rendering the incorporating crosslinking reaction more efficient and more likely. It has been proposed that transient and circulating proteins in the Amadori form can become permanently crosslinked to tissue structural proteins, conferring additional and potentially synergistically deleterious properties to the glycated tissue matrix. For example, it has been suggested that such incorporation of albumin into tissues could instigate chronic edema and fibrosis due to the high oncotic strength of albumin. Thus, the mechanism of glycation-mediated fixation of infiltrated circulating proteins is likely to be relevant to clinical implants comprised of bioprosthetic tissues and biomaterials.

Current BHV Testing Paradigms

Both current FDA standards for approval of BHV models and the internal research and development testing undertaken by BHV manufacturers are guided by recommendations set forth by the three components of ISO 5840. Current testing standards call for the evaluation of valve performance under physiologic conditions as well as the evaluation of gross structural durability over a reasonable lifetime of cardiac cycles. Testing standards cover both testing conditions and evaluation endpoints. Physiologically-relevant valve performance data is obtained using dual-chamber mechanical fluid-pulsing systems capable of achieving and maintaining the full breadth of physiological pressure, flow, and pulse rate conditions. BHV are mounted between the chambers of these so-called “pulse duplicators” and endpoint data is acquired during pulse cycles. The endpoint parameters recommended by ISO and utilized in FDA requirements define performance ranges and acceptable minimum performance cutoffs for effective orifice area, regurgitation fraction, valve pressure gradients, and cardiac cycle waveforms for flow and pressure. Valve durability is assayed on cyclical fatigue testers. Fatigue testers operate on the same mechanical principles as pulse duplicators but cycle at tremendous rates (generally 1,500-2000 cycles per minute) in order to recapitulate numbers of valve opening and closing cycles experienced during many years of clinical implantation in relatively short periods of time. ISO and FDA standards require BHV to maintain structural integrity and performance characteristics through 200,000,000 cycles in fatigue testing.

The ideal medium for valve testing is human blood, which is the fluid in which BHV are constantly immersed during clinical implantation. However, it is not feasible to utilize blood for industrial valve testing due to both practical and technical reasons. Yet, the various physical and biochemical characteristics of blood impinge on valve performance and durability, as described above. Nonetheless, ISO recommendations and FDA requirements essentially ignore these considerations, taking into account only the salt content and buffered nature of physiological fluids by stipulating saline or phosphate-buffered saline as testing media. Current standards also consider physiologic temperature, stipulating testing at 37° C. Valve manufacturers account for blood viscosity by adding glycerin to these solutions, and even this is above and beyond the existing ISO 5840 standards and recommendations. The effects on valve performance and durability of blood biochemistry and blood component infiltration are not currently considered by testing recommendations, standards, or practices.

Utilization of Blood Protein-containing and Glycation-capable Solutions in Bioprosthetic Valve Testing

Compositions and methods for performance and durability testing of bioprosthetic heart valves (BHV) as part of the industrial process of their manufacture and approval is detailed below. Specifically, the methods involve the utilization of solutions containing serum albumin and in some embodiments glycation precursors as opposed to simple saline or water—which are currently the only fluids used—for their testing.

The inventors have determined, through studies on the degeneration of clinically-implanted bioprosthetic heart valves, that infiltration and incorporation of serum albumin into the valve tissue occurs in clinical valves, modifies tissue properties/valve performance, and contributes to valve degeneration. Albumin is by far the most abundant protein in the blood—accounting for over half of all blood proteins by mass; therefore, it is inherently uniquely important with regard to blood components in this respect. The inventors discovered that the bioprosthetic tissues from which these valves are manufactured have no resistance to the infiltration of albumin from surrounding fluid; as a result, the inventors have shown that albumin and glycation precursors begin to infiltrate into the tissue essentially immediately upon exposure to a fluid containing it (i.e., blood, in the clinical context) and throughout the depth of the tissue, both in vitro and in vivo. Further, the inventors have established that albumin can be permanently incorporated into the valve tissue by glycation via physiologically-relevant precursors, such as sugars and sugar-derived dialdehydes. In this context, the inventors established that albumin incorporation has both glycation-related and glycation-independent effects on valve tissue and performance. The presently disclosed manufacturing and industrial testing methods, as well as methods for testing the efficacy of anti-glycation compounds, address these considerations for commercial BHV and related biomaterial implant performance and durability.

First, the inventors have proven that exposure to albumin alone at physiological blood protein concentration (5%) reliably increases the “dry” mass of valve leaflet tissue by 4.5-5%. This is highly meaningful to clinical valve performance for reasons of biomechanics. Moving a greater mass inherently increases the biomechanical stress that a tissue is subjected to during biomechanical activity. Thus, moving 4.5-5% more mass than expected in the biomechanical activity of the valve during every heartbeat is expected to accelerate biomechanical fatigue and associated tissue structural degeneration—particularly compounded over the hundreds of millions of beats accomplished in a decade of clinical implantation. Beyond even this, albumin has a uniquely high oncotic pressure, meaning that it pulls water molecules into tight association with itself and hyperhydrates tissues into which it has infiltrated or incorporated. This is why albumin hyperinfiltration is associated with edema in body tissues. Thus, the actual mass of a valve leaflet in a clinical setting is expected to increase even more than the 5% dry mass change observed.

Further, exposure to albumin and 50 mM glyoxal, a physiologically-relevant glycating agent, leads to a galloping increase in the accumulated dry mass with increasing incubation time, gradually reaching over 13% after 28 days of incubation. This suggests that permanent incorporation of infiltrated albumin via physiological biochemical crosslinking exacerbates over time the tissue modification and biomechanically-meaningful mass increase that begins essentially immediately upon albumin exposure.

Second, albumin has particular properties that cause it to modify valve tissue functionality—including biomechanical performance—and instigate structural degeneration. Its high oncotic pressure is one of these properties. Another is that albumin is a globular (approximately spherical) protein, compared to the thin and elongated fibrous nature of BHV tissue collagen, which is arranged in stacked layers of organized, parallel fibers. Further, while collagen and glycosaminoglycans—two proteins accounting for nearly all of the BHV tissue material—are positively-charged, albumin is negatively-charged. This charge difference encourages close interaction of the proteins and modification of the stereoelectronic environment of the tissue proteins. All of these properties can be expected to disrupt BHV tissue molecular organization by sterically, stereoelectronically, and osmotically disrupting collagen network alignment, normal interactions among tissue proteins, and resultant dissipation of forces during valve biomechanical activity. The inventors have demonstrated, using second-harmonic generation imaging, that collagen network alignment and fiber properties are disrupted over time via exposure to 5% albumin alone—and more so with concomitant exposure to glycation precursor.

Third, in the context of glycation, infiltrated albumin molecules and can mediate crosslinks between distant collagen fibers, eliciting larger-scale tissue stiffening than the local stiffening due to crosslinking of directly-adjacent collagen fibers. Clinically, 1-10% of serum albumin circulating in the blood of normoglycemic people at any given time is in the Amadori form, which is an intermediate state in the glycation process that is primed for further condensation to generate glycation crosslinks that can permanently bind the albumin to the structural proteins of the valve tissue. In diabetics, the proportion reaches 30%. Incorporation of albumin infiltrated into solid tissues from body fluids via condensation of Amadori modifications into AGE crosslinks will both rigidify the tissue and compound the disruptive effects of albumin arising from the properties discussed above on the collagen network and the coherence of the extracellular matrix proteins.

BHV manufacturers routinely perform cyclical fatigue testing and functional modeling using pulse duplicators. However, no valve manufacturers are cognizant of the above information and do not take the inherent effects of albumin on the properties evaluated in these tests into account. FDA standards for approval of valves are based on ISO recommendations, and both of these stipulate only saline or phosphate-buffered saline for these tests. Valve manufacturers only employ these two solutions for these tests and do not employ anything involving albumin in these tests.

Based on the results disclosed herein, valve manufacturers are testing performance under conditions that are not reflective of their clinical performance, and therefore testing should be done in albumin-containing solutions in order to take into account the inherent and immediate effects of this most abundant blood protein on clinical valve function. Therefore, the inventors have discovered that the industrial process design of performing pulse duplicator performance testing and fatigue testing should be performed in solutions (which may be saline or phosphate-buffered saline) containing serum albumin (for example 1-10%) and/or glycation precursors and/or glycated albumin (for example at 1-10% of the total albumin used) at physiologic and modeling concentration ranges.

The present disclosure constitutes paradigms for BHV testing that account for the effects of key blood component infiltration into and reaction with BHV. Namely, the present disclosure enables consideration of BHV tissue susceptibility to both albumin infiltration as well as glycation in valve testing and evaluate the effects of such on valve physiologic performance and durability. It is impractical to utilize blood itself for valve testing to fully encompass the complexity that a clinically-implanted valve is exposed to; however, utilizing albumin-containing solutions will account for the majority of protein infiltration capacity while also contributing more physiologic viscosity to the testing fluid.

Additionally, because albumin contributes the majority of oncotic pressure in body fluids and is the most prolific binder of other blood molecules, including lipids and calcium, albumin-containing solutions easily can be used to test the significance of these other factors to valve performance and durability by augmentation of the albumin solutions with these other molecules. Thus, the present disclosure provides utilization in BHV performance and durability testing of non-buffered salt solutions, including but not limited to saline, and buffered solutions, including but not limited to physiologically-relevant solutions such as phosphate-buffered saline and experimentally useful in vitro buffer systems such as HEPES, containing dissolved albumin at concentrations representing physiological albumin concentration ranges observed in patients as well as concentrations wherein albumin is further employed as a proxy for total blood protein concentration, oncotic pressure, viscosity, or other properties. Initial buffer solutions can be calibrated to pH 7.4, pH 7.35, and pH 7.45 in order to represent standard normal blood pH as well as the cutoff pH points for acidemia and alkalemia, respectively. Additionally, buffer solutions can be calibrated to pH's ranging between 6.8 and 7.8 at 0.1 unit intervals in order to cover the range of acidemia and alkalemia conditions considered to be compatible with life. In various embodiments, buffer solutions can be used including buffers having a pH of 6.8, 6.9, 7.0, 7.1, 7.2, 7.3, 7.4, 7.5, 7.6, 7.7, or 7.8, or any range that includes one or more of the foregoing. Additional embodiments of the present disclosure include the utilization of other blood proteins, including, but not limited to, immunoglobulins, hemoglobin, and calcium- and lipid-binding proteins, in place of albumin, either individually or in any combination with or without albumin. A further embodiment of the present disclosure is the pre-treatment of valves with such solutions prior to testing in other solutions, including, but not limited to, the current standard solutions. Specifics of the subject solutions and additional considerations and embodiments are discussed below.

Serum albumin levels are subject to tight homeostatic regulation. The reference range of normal serum albumin levels in healthy human individuals is 33-52 mg/L of plasma (Transfus. Med. Hemother. 36:399-407, 2009).

Hyperalbuminemia is a state of elevated albumin levels that is most often transient and due to dehydration; however, nutrition-dependent but sustained hyperalbuminemia has also been noted in individuals following high-protein diets as part of personal physical fitness regimens. The state of hyperalbuminemia can exhibit albumin levels raised near the 60 mg/L mark. Albuminemia—abnormally low levels of serum albumin—is more common and can be sustained for long periods, due to inflammatory circumstances and nutritional state. Analbuminemia is a rare, genetically-driven condition in which circulating albumin levels are nearly undetectable in human individuals. Finally, hyperoncotic infusion solutions containing up to about 25% albumin are utilized for volume expansion during surgeries and for blood loss. Thus, circulating albumin concentrations from trace amounts (>0%) to about 25% can be considered physiologically relevant and represent the range of albumin concentrations considered for use in the methods of the present disclosure.

The effects of glycation on valve performance and durability are also considered in the present disclosure. Glucose is the most abundant blood sugar and circulating glycation precursor. Glucose levels fluctuate throughout the day and in response to eating or physical activity; however, circulating glucose levels generally range between 3.9-7.8 mM in normoglycemic, healthy individuals. In diabetics, this level can spike as high as roughly 100 mM; however, such acute ketoacidosis is extremely critical and cannot be sustained. Immediate emergency medical attention is recommended upon a reading of 16.6 mM glucose (3 g/L), but concentrations in the range of 10-16.6 mM can be sustained to some degree. While sugars are much more abundant than oxidized sugar-derived carbonyl-containing small molecules, these derivative molecules are much more reactive for glycation than sugars. Additionally, different glycation precursors generate different glycation products and intermediates, all contributing to the vast array of glycation products formed physiologically. Thus, all glycation precursors must be considered with respect to overall glycation. Physiologic concentrations of circulating sugar-derived reactive carbonyls are orders of magnitude lower than concentrations used thus far to validate their effects on valve performance in a pulse duplicator—generally occupying the low- and sub-micromolar concentration space. However, such elevated in vitro concentrations are useful for modeling in reasonable testing timescales the effects on valve performance and durability that occur due to years of physiologically-imposed glycation. Additionally, both physiologic and supra-physiologic concentrations of glycation precursors in conjunction with circulating protein infiltration affect protein incorporation and resultant modification of valve properties by exposure to these proteins and can be used to model real-time effects and long-term effects, respectively. Thus, the present disclosure considers the full range of glycation precursors from trace concentrations to in vitro modeling concentrations on the order of 10-500 mM for each glycation precursor. Glycation precursors include, but are not limited to, glucose, fructose, sucrose, galactose, ribose, maltose, dextrose, glyoxal, methylglyoxal, 3-deoxyglucosone, glycolaldehyde, and ascorbic acid. More broadly, the considered precursor classes can be described as sugars (including hexoses and pentoses), sugar-derived reactive aldehydes and ketones, and small molecule metabolites and vitamins (such as ascorbic acid). In a further embodiment, precursors of lipoxidation may also be considered.

Further, the synergy between serum albumin/blood protein infiltration and glycation on BHV performance and durability can be accounted for by the utilization of solutions containing both blood proteins and elements of glycation.

By encompassing ranges and combinations of levels of serum albumin, alternative circulating proteins, glycation precursors, glycated albumin, and/or glycated alternative proteins reflective of the blood biochemistry of BHV patient profiles exhibiting diverse variations and comorbidities, such as diabetes, the presently disclosed testing technology incorporates elements of personalized medicine. In particular, the susceptibility of albumin to glycation is known to directly affect its incorporation into tissues, causes it to aggregate, modifies its molecular binding properties, and enables it to be a powerful stimulator of inflammation. As stated, a range from about 1% to about 10% of serum albumin circulating in a human individual at any given time is glycated, with nearly all of it being in the important Amadori form. The present disclosure accounts for the contribution this will have on BHV valve performance and durability immediately upon and all through clinical implantation via the facilitated incorporation of albumin into BHV tissue through glycation crosslinking. Testing solutions disclosed herein include solutions spanning both physiologically-relevant and in vitro modeling proportions of Amadori- and endpoint-glycated albumin versus total albumin, from 0% glycated albumin to 100% glycated albumin. In an additional embodiment, other blood proteins will be used in place of and/or in addition to albumin. Preparations of albumin in various forms of glycation, including but not limited to Amadori-albumin and albumin driven to endpoint glycation by the various glycation precursors, will be added to unglycated albumin to generate solutions of glycated-to-unglycated albumin at incremental proportions. Base serum albumin can be obtained via purification from human blood, animal blood including, but not limited to, bovine, or recombinantly expressed and purified from artificial host species including, but not limited to, bacteria, yeast, and plants. Finally, therefore, the present disclosure includes the design and preparation of all of the solutions described above for testing and pretreatment of BHV and related biomaterials.

A general flow chart outlining the testing method is shown below (exemplified by glutaraldehyde-fixed bovine pericardium and bioprosthetic heart valves manufactured from other materials, such as glutaraldehyde-fixed porcine aortic valve and bovine jugular vein).

Solution Implementation During Testing
Fill testing device (pulse duplicator, cyclical fatigue tester, etc) with
augmented testing solution (containing e.g. albumin and/or glycation
precursors)
↓
Pre-warm the testing chambers and solutions to 37° Celsius and set flow,
pressure, and cycle rate parameters to desired conditions
↓
Mount BHV, allow it to equilibrate to temperature (and to testing solution,
if desired), and recalibrate conditions, remove bubbles, etc.
↓
Run test
↓
Acquire testing endpoints (effective orifice area, pressure gradient across
valve, opening/closing rates, leaflet motion data, coaptation data, etc
↓
Dismount and retain BHV for additional experimental endpoints
(immunohistochemistry, tissue biomechanical tests, additional
exposures/treatments etc

Solution Implementation as Pre-Treatment
Fill testing device (pulse duplicator, cyclical fatigue tester, etc) with
augmented testing solution (containing e.g. albumin and/or glycation
precursors)
↓
Rinse BHV (either simply to remove excess solution or to allow for
diffusion out of infiltrated molecules in saline or alternative solution
as desired for subsequent testing)
↓
Prepare testing device using current standard or augmented testing solution
↓
Mount BHV, Run test, and acquire endpoints
↓
Dismount and retain BHV for additional experimental endpoints
(immunohistochemistry, tissue biomechanical tests, additional
exposures/treatments, etc

Comparison of the current testing methodologies with the augmented testing methodologies for various valve physiologic environment considerations reveals that while both testing methodologies evaluate valve performance at physiological temperature, valve performance/durability in water based salt solution, valve performance/durability at physiologically-relevant pH, and the effect of blood-like viscosity on valve performance/durability, only the augmented testing methodologies evaluate the effects of blood chemistry on valve performance/durability, incorporates known mechanisms of tissue modification/SVD, and is able to model the effects of comorbidities and address personalized medicine concerns.

General Testing Solution Recipes

### Basic Albumin-augmented Testing Solutions.

Base buffered salt solution (pH 7.4 and others as indicated in accompanying text).

Serum albumin dissolved at about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%, or any concentration greater than 0% up to about 25% of the total protein concentration. Alternative proteins may be used in addition to or in place of serum albumin at similarly-graded concentration levels based on their established physiologically-relevant concentration ranges or in vitro modeling concentrations.

### Basic Glycation-augmented Testing Solutions.

Base buffered salt solution (pH 7.4 and others as indicated in accompanying text).

Glycation precursors as identified in accompanying text, dissolved or mixed at concentrations ranging from about 1 nM to about 5 μM to represent physiologically-relevant levels (as detailed herein) and up to about 500 mM for in vitro modeling purposes. For example, glycation precursors can be mixed at concentrations of about 1 nM, about 2 nM, about 3 nM, about 4 nM, about 5 nM, about 6 nM, about 7 nM, about 8 nM, about 9 Nm, about 10 nM, about 20 nM, about 30 nM, about 40 nM, about 50 nM, about 60 nM, about 70 nM, about 80 nM, about 90 nM, about 100 nM, about 110 nM, about 120 nM, about 130 nM, about 140 nM, about 150 nM, about 160 nM, about 170 nM, about 180 nM, about 190 nM, about 200 nM, about 210 nM, about 220 nM, about 230 nM, about 240 nM, about 250 nM, about 260 nM, about 270 nM, about 280 nM, about 290 nM, about 300 nM, about 310 nM, about 320 nM, about 330 nM, about 340 nM, about 350 nM, about 360 nM, about 370 nM, about 380 nM, about 390 nM, about 400 nM, about 410 nM, about 420 nM, about 430 nM, about 440 nM, about 450 nM, about 460 nM, about 470 nM, about 480 nM, about 490 nM, about 500 nM, about 510 nM, about 520 nM, about 530 nM, about 540 nM, about 550 nM, about 560 nM, about 570 nM, about 580 nM, about 590 nM, about 600 nM, about 610 nM, about 620 nM, about 630 nM, about 640 nM, about 650 nM, about 660 nM, about 670 nM, about 680 nM, about 690 nM, about 700 nM, about 710 nM, about 720 nM, about 730 nM, about 740 nM, about 750 nM, about 760 nM, about 770 nM, about 780 nM, about 790 nM, about 800 nM, about 810 nM, about 820 nM, about 830 nM, about 840 nM, about 850 nM, about 860 nM, about 870 nM, about 880 nM, about 890 nM, about 900 nM, about 910 nM, about 920 nM, about 930 nM, about 940 nM, about 950 nM, about 960 nM, about 970 nM, about 980 nM, about 990 nM, about 1 μM, about 2 μM, about 3 μM, about 4 μM, about 5 μM, about 6 μM, about 7 μM, about 8 μM, about 9 μM, about 10 μM, about 20 μM, about 30 μM, about 40 μM, about 50 μM, about 60 μM, about 70 μM, about 80 μM, about 90 μM, about 100 μM, about 110 μM, about 120 μM, about 130 μM, about 140 μM, about 150 μM, about 160 μM, about 170 μM, about 180 μM, about 190 μM, about 200 μM, about 210 μM, about 220 μM, about 230 μM, about 240 μM, about 250 μM, about 260 μM, about 270 μM, about 280 μM, about 290 μM, about 300 μM, about 310 μM, about 320 μM, about 330 μM, about 340 μM, about 350 μM, about 360 μM, about 370 μM, about 380 μM, about 390 μM, about 400 μM, about 410 μM, about 420 μM, about 430 μM, about 440 μM, about 450 μM, about 460 μM, about 470 μM, about 480 μM, about 490 μM, about 500 μM, about 510 μM, about 520 μM, about 530 μM, about 540 μM, about 550 μM, about 560 μM, about 570 μM, about 580 μM, about 590 μM, about 600 μM, about 610 μM, about 620 μM, about 630 μM, about 640 μM, about 650 μM, about 660 μM, about 670 μM, about 680 μM, about 690 μM, about 700 μM, about 710 μM, about 720 μM, about 730 μM, about 740 μM, about 750 μM, about 760 μM, about 770 μM, about 780 μM, about 790 μM, about 800 μM, about 810 μM, about 820 μM, about 830 μM, about 840 μM, about 850 μM, about 860 μM, about 870 μM, about 880 μM, about 890 μM, about 900 μM, about 910 μM, about 920 μM, about 930 μM, about 940 μM, about 950 μM, about 960 μM, about 970 μM, about 980 μM, about 990 μM, about 1 mM, about 2 mM, about 3 mM, about 4 mM, about 5 mM, about 6 mM, about 7 mM, about 8 mM, about 9 mM, about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, or about 500 mM, or any ranges or subranges of the foregoing.

Amadori-Albumin-augmented testing solutions.

Base buffered salt solution (pH 7.4 and others as indicated in accompanying text).

Total albumin dissolved at the concentrations indicated for base albumin-augmented testing solutions, including:

Unmodified serum albumin (produced either via recombinant expression strategies or chromatographic removal of modified serum albumin from isolated stocks), concentration adjusted to account for the ratio of modified albumin; and

Amadori-albumin dissolved or mixed from stock at final concentrations of about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35%, or any concentration greater than 0% to about 35% of the total concentration.

### Endpoint-glycated (AGE)-Albumin-augmented Testing Solutions.

Base buffered salt solution (pH 7.4 and others as indicated in accompanying text).

Total albumin dissolved at the concentrations indicated for base albumin-augmented testing solutions, including:

Unmodified serum albumin (produced either via recombinant expression strategies or chromatographic removal of modified serum albumin from isolated stocks), concentration adjusted to account for the ratio of modified albumin; and

AGE-albumin dissolved or mixed from stock at final concentrations of about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35%, or any concentration greater than 0% to about 35% of the total concentration.

### Calcification+Albumin Augmented Testing Solutions.

Base buffered salt solution (pH 7.4 and others as indicated in accompanying text).

Total serum albumin dissolved at about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%, or any concentration greater than 0% to about 25% of the total concentration. Alternative proteins may be used in addition to or in place of serum albumin at similarly-graded concentration levels based on their established physiologically-relevant concentration ranges specific to both normal and particular disease blood levels.

Physiologically-relevant and established in vitro modeling calcium salts at physiologically-relevant and in vitro modeling concentrations (useful in that albumin is a known calcium-binding protein and thus likely influences calcification processes).

Auxiliary molecules noted in the process of calcification.

### Lipids+Albumin Augmented Testing Solutions.

Base buffered salt solution (pH 7.4 and others as indicated in accompanying text).

Total serum albumin dissolved at about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%, or any concentration greater than 0% to about 25% of the total concentration. Alternative proteins may be used in addition to or in place of serum albumin at similarly-graded concentration levels based on their established physiologically-relevant concentration ranges specific to both normal and particular disease blood levels.

Physiologically-relevant and established in vitro modeling lipids and lipid complexes at physiologically-relevant and in vitro modeling concentrations (useful in that albumin is known to be a lipid-binding protein and thus likely influences established lipid infiltration phenomena).

Auxiliary molecules noted in the process of calcification.

### Oxidizers+Albumin Augmented Testing Solutions.

Base buffered salt solution (pH 7.4 and others as indicated in accompanying text).

Total serum albumin dissolved at about 0.05%, about 1%, about 2%, about 3%, about 4%, about 5%, about 6%, or about 7%, or any concentration greater than 0% to about 7% of the total concentration (broadly physiological range). Alternative proteins may be used in addition to or in place of serum albumin at similarly-graded concentration levels based on their established physiologically-relevant concentration ranges specific to both normal and particular disease blood levels.

Physiologically-relevant and established in vitro modeling lipids and lipid complexes at physiologically-relevant and in vitro modeling concentrations.

Auxiliary molecules noted in the process of calcification.

Useful in that albumin is known to be an antioxidant protein and thus likely influences established valve oxidation phenomena

### Valve Technology Testing Solutions.

Base buffered salt solution (pH 7.4 and others as indicated in accompanying text, or at pH's and salt/buffer conditions relevant to the treatment technology).

Relevant macromolecules and small molecules to the mechanism intending to be addressed by the technology (e.g., glycation precursors for valve glycation).

Components of the interventional technology or drug at operating concentrations.

### Personalized Medicine Testing Solutions.

Base buffered salt solution (pH 7.4 and others as indicated in accompanying text).

Total serum albumin dissolved at about 0.05%, about 1%, about 2%, about 3%, about 4%, about 5%, about 6%, about 7%, or any concentration greater than 0% to about 7% (broadly physiological range). Alternative proteins may be used in addition to or in place of serum albumin at similarly-graded concentration levels based on their established physiologically-relevant concentration ranges specific to both normal and particular disease blood levels.

Unmodified proteins (produced either via recombinant expression strategies or chromatographic removal of modified serum albumin from isolated stocks), concentration adjusted to account for the ratio of modified albumin.

Modified proteins (e.g., Amadori- and/or endpoint-glycated albumin) dissolved or mixed from stock at ratios reflective of the relevant disease states (e.g., 10%-35% of total serum albumin as glycated albumin for diabetes).

Additional auxiliary macromolecules and small molecules relevant to established and/or hypothesized disease processes, such as metal ions or enzyme cofactors.

Optionally, established or experimental interventional molecules and/or solutions (e.g., medicines, drugs, natural products) relevant to the disease states or personalized medical attributes being evaluated.

Generation of Proprietary Solutions

### Stock Protein Preparation:

Human proteins or animal alternatives can be recombinantly expressed in established organism systems and purified and/or isolated from natural sources/physiological fluids according to standard protocols. Protein solutions are 0.22 μm-diameter sterile filtered and maintained in sterility either lyophilized, frozen, or at 4° C.

### Glycation Precursors:

Due to their reactive nature, glycation precursors should be provided in separate stock bottles—either pre-aliquoted in specific doses or as bulk solution or powder—and added to the test/pre-incubation solutions upon use.

### Amadori-Protein Stock Preparation:

1. Human or animal proteins derived as above are incubated at preparative concentrations (e.g., 5%-25% for serum albumin) in PBS or specific protein-compatible reaction buffer with sugars (e.g., glucose, fructose, galactose) at physiological or accelerated in vitro modeling concentrations (e.g., 200 mM for glucose) at 37° C. for intermediate lengths of time (e.g., 4 days to 3 weeks).

2. Size-exclusion “desalting” chromatography is applied in order to separate protein from unreacted glycation precursor. Reaction solutions containing protein, glycated protein, and unreacted glycation precursor as well as any small molecule byproducts are loaded onto and run through gel filtration chromatography columns. Protein peak(s) are monitored by UV absorption and removal of glycation precursors and related small molecules will be confirmed by mass spectrometry.

3. Protein-containing fractions from Step 2 are loaded onto affinity chromatography columns loaded with beads conjugated to antibodies specific for Amadori-modified protein of interest and/or phenylboronate agarose resin. For example, in the case of serum albumin, beads conjugated to A717 antibody (Exocell), which specifically recognized Amadori-albumin, can be used.
a. Note that antibodies specific for Amadori-modified forms of proteins of interest are often not available for specific proteins. In such cases, antibodies or resins (such as phenylboronate agarose) that generally recognize Amadori adducts can be used. Purity of the final preparation should not be affected, since the starting solutions should already only contain pure protein of interest.
b. In cases of the above where purity issues arise, a second affinity column containing beads conjugated to antibody specific for the protein of interest can be used.

4. Purified modified-protein solutions will be 0.22 μm sterile filtered and stored as stock protein.

### Endpoint Glycated Protein Stock Preparation:

1. Human or animal proteins derived as above are incubated at preparative concentrations (e.g., about 5%-about 25% for serum albumin) in

PBS or specific protein-compatible reaction buffer with glycation precursors (e.g., sugars, sugar-derived dialdehydes) at physiological or accelerated in vitro modeling concentrations (e.g., about 50 mM for glyoxal) at 37° C. for lengths of time sufficient to allow for the resolution of any known or likely glycation intermediates to endpoint glycation products (e.g., about 2 weeks to about 1 month for sugar-derived dialdehydes and about 2 months to about 6 months for sugars).

2. Size-exclusion “desalting” chromatography is applied in order to separate protein from unreacted glycation precursor. Reaction solutions containing protein, glycated protein, and unreacted glycation precursor as well as any small molecule byproducts are loaded onto and run through gel filtration chromatography columns. Protein peak(s) are monitored by UV absorption and removal of glycation precursors and related small molecules is confirmed by mass spectrometry.

3. Protein-containing fractions from Step 2 are loaded onto affinity chromatography columns loaded with beads conjugated to antibodies specific for AGE-modified protein of interest. Note that antibodies specific for glycation-modified forms of proteins of interest are often not available for specific proteins. In such cases, antibodies that generally recognize AGE adducts (e.g., N-carboxymethyl lysine, also known as CML) can be used. Purity of the final preparation should not be affected, since the starting solutions should already only contain pure protein of interest.
a. In cases of the above where purity issues arise, a second affinity column containing beads conjugated to antibody specific for the protein of interest can be used.

4. Purified modified-protein solutions are 0.22 μm sterile filtered and stored as stock protein.

### Molecules Related to Additional Processes and Mechanisms of Interest to Valve Testing and SVD (e.g., Lipid Infiltration, Calcification):

1. Human molecules or animal alternatives can be recombinantly expressed in established organism systems and purified and/or isolated from natural sources/physiological fluids according to standard protocols. Solutions are 0.22 μm-diameter sterile filtered and maintained in sterility either lyophilized, frozen, or at 4° C.

2. Specific buffer and stabilizer recipes may be required for individual types of molecules and preparations (e.g., circulating lipid particles) and are defined as relevant.

### Working Solutions.

Stock solutions and/or powders prepared and/or maintained as above are dissolved/mixed in phosphate-buffered saline or appropriate buffer and salt solution under sterile conditions to final concentrations and ratios.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain an effective amount of an anti-glycation agent described herein, which can be in purified form, together with a suitable amount of carrier so as to provide the form for proper administration to the subject.

The term “formulation” refers to preparing an agent in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Kits

Also provided are kits. Such kits can include an agent or composition described herein and, in certain embodiments, instructions for testing. Such kits can facilitate performance of the methods described herein. When supplied as a kit, the different components of the composition can be packaged in separate containers and admixed immediately before use. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to another component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

What is claimed is:

1. A method of testing performance or durability of a bioprosthetic heart valve, the method comprising testing the performance or durability of the bioprosthetic heart valve in a pulse duplicator or cyclical fatigue tester in a solution containing serum albumin or a glycation precursor.

2. The method of claim 1, wherein performance of the bioprosthetic heart valve is tested in a pulse duplicator.

3. The method of claim 1, wherein durability of the bioprosthetic heart valve is tested in a cyclical fatigue tester.

4. The method of claim 1, wherein the serum albumin or glycation precursor is present in the solution in a physiological concentration.

5. The method of claim 1, wherein in the glycation precursor is glucose, fructose, galactose, sucrose, maltose glyoxal, methylglyoxal, 3-deoxyglucoson, glycolaldehyde, ascorbic acid, or any other physiologically relevant precursor.

6. The method of claim 1, wherein the solution comprises from greater than zero to about 25% serum albumin.

7. The method of claim 1, wherein the serum albumin is human serum albumin.

8. The method of claim 1, wherein greater than zero to about 35% of the serum albumin is glycated.

9. The method of claim 1, wherein an alternative physiologically circulating protein is substituted for serum albumin at correspondingly relevant concentrations.

10. The method of claim 1, wherein body fluid components associated with alternative biochemical pathways and disease processes are substituted for glycation precursors or added with glycation precursors for the modeling of the effects of said alternative pathways and processes on valve and bioprosthetic tissue function and durability.

11. The method of claim 10, wherein the body fluid components associated with alternative biochemical pathways and disease processes are metal ions, cholesterol and lipid nanoparticles, or enzyme cofactors.

12. The method of claim 10, wherein the alternative disease process is calcification.

13. The method of claim 10, wherein the solution containing body fluid component molecules is applied as a pre-treatment of the bioprosthetic heart valve or bioprosthetic tissue before testing.

14. A kit for testing performance or durability of a bioprosthetic heart valve, comprising:
a buffered or non-buffered saline solution; and
serum albumin.

15. The kit of claim 14, further comprising:
a glycation precursor.

16. The kit of claim 14, wherein the serum albumin is human serum albumin.

17. The kit of claim 14, wherein greater than zero to about 35% of the serum albumin is glycated.

18. The kit of claim 14, further comprising a body fluid component associated with alternative biochemical pathways or disease processes.

19. The kit of claim 18, wherein the body fluid component associated with alternative biochemical pathways and disease processes is a metal ion, a cholesterol and lipid nanoparticle, or an enzyme cofactor.

20. The kit of claim 18, wherein the alternative disease process is calcification.
